(19) United States
(12) Patent Application Publication
(10) Pub. No.: US 2007/0072251 A1
(43) Pub. Date: Mar. 29, 2007

(54) In vivo imaging using peptide derivatives

(75) Inventors: Kalevi Kairemo, Helsinki (FI); Sami Kaukinen, Helsinki (FI); Heli Valtanen, Soderkulla (FI)

Correspondence Address: BIRCH STEWART KOLASCH & BIRCH, PO BOX 747, FALLS CHURCH, VA, 22040-0747, US

(73) Assignee: CTT Cancer Targeting Technologies OY, Helsinki (FI)

(21) Appl. No.: 10/554,996

(22) PCT Filed: May 3, 2004
(86) PCT No.: PCT/FI04/00265

§ 371 Date: Aug. 7, 2006

(30) Foreign Application Priority Data
May 2, 2003 (FI) 20030664

(51) Int. Cl. G01N 33/574; A61K 38/10

(52) U.S. Cl. 435/7.23; 514/15

(57) ABSTRACT

The present invention relates to the use of phage display LLG peptide derivatives as tumor targeting agents for diagnostic purposes, and to a method for targeting and imaging tumors and infections/inflammation. A diagnostic composition comprising said peptide derivatives is also disclosed.

### FIELD OF THE INVENTION

The present invention relates to phage display LLG peptide derivatives as tumor targeting agents and as imaging agents for diagnostic purposes, and to a method for targeting and imaging tumors and infections/inflammation. A diagnostic composition comprising said peptide derivatives is also disclosed.

### BACKGROUND OF THE INVENTION

Despite of important advantages in the therapy of acute myeloid leukemia (AML), the majority of patients will die from their disease. Approximately half of the children with AML can be cured, but little progress has led to gradual improvement in long-term survival of older adults with AML. Treatments in AML are hard, including high doses of cytotoxic agents and allogenic stem cell transplants, but still majority of adults relapse. In a few years new strategies have arisen to improve the cure of AML, such as unmodified antibody or cell based immunotherapies. However, diseases such as AML, for which the outcome remains poor, should be treated on clinical trials whenever possible.

The integrin CD11 has been correlated with a poor prognosis of the AML. A bioactive peptide obtained recently by phage display is a specific ligand to the leukocyte β₂ integrins. By panning on purified α_{M}β₂ integrin (CD11/CD18) a novel nonapeptide CPCFLLGCC (LLG) was isolated, which is dependent on two disulfide bridges that constrain the peptide structure (see WO 02/072618, which is incorporated herein by reference).

A variety of peptide based radioligands are currently under development for in vivo therapeutic and diagnostic strategies, including bombesin, gastrin/cholecystokinin, and neurotensin, which are receptors expressed on common cancers, and Arg-Gly-Asp peptides, which, because they bind to receptors expressed on newly formed blood vessels, can be targeted to many common tumors.

Inflammation is a defence mechanism, which consists of release of proinflammatory mediators, selectin mediated leukocyte adhesion to the endothelial cells of surrounding blood vessels, activation of specific leukocyte integrins, firmer adhesion by interaction of integrin and intercellular adhesion molecules (ICAMs) and leukocyte extravasation.

Integrins are involved in a wide range of activities concerning the intercellular communication, and they are grouped into sub-families according to distinct β subunits. Leukocytes express only β₂ integrins. Four members of the β₂ integrin family are α_{L}β₂ or CD11a/CD18, α_{M}β₂ or CD11b/CD18 or Mac-1, α_{X}β₂ or CD11c/CD18 and α_{D}β₂ or CD11d/CD18. ICAMs are the major ligands of the β₂ integrin family, and they have a common recognition sequence LLG, which is favored by α_{M}β₂ integrin. α_{M}β₂ integrin is involved in immune reactions by binding iC3b-coated erythrocytes, mediating the adherence and phagocytosis of myeloid cell, enhancing NK cell activity. α_{M}β₂ integrin is involved in macrophage-microorganism interactions and it also mediates cell adhesive interactions on myeloid cells. α_{M}β₂ has other ligands including factor X and fibrinogen.

As stated above, a bioactive peptide obtained recently by phage display is a specific ligand to the leukocyte β₂ integrins. By panning on purified α_{M}β₂ integrin (CD11/CD18) a novel nonapeptide CPCFLLGCC (LLG) was isolated which is dependent on two disulfide bridges that constrain the peptide structure. Studies with differentially cyclized peptides indicated that a particular disulfide configuration is more active than another one. The preferred peptide for the use according to the present invention is the peptide with one disulfide bond between the C1 and C8 cysteines, and a second disulfide bond between the C3 and C9 cysteines. The peptide inhibits the α_{M}β₂ integrin-mediated leukocyte cell adhesion and binds to the cation-sensitive I-domain of the integrin a subunit. The NMR structures of the two LLG conformers were determined and the more active conformer serves as a lead for development of potential anti-inflammatory agents and leukemia cell-targeting compounds. Here, we explore the possibilities to use LLG as an inflammation and tumor targeting and imaging agent. LLG can also be pegylated to improve its therapeutic effect. We will also have a project in which the LLG is crystallized with the complex with I-domain and design an organic analogue of the peptide. We will also evaluate the potential of the analogue with these animal models described here. The LLG can also function as a therapeutic agent on surface of liposome. Using liposome we can modify the pharmacokinetics and dynamics of the peptide.

The use of LLG or LLG-PEG as an imaging agent for diagnostic purposes is described. This work describes also a new strategy to target AML cells with a peptide based method which could be utilized in a targeted therapy.

### SUMMARY OF THE INVENTION

In this invention we demonstrate the tumor and infection targeting properties of LLG peptide derivatives. LLG is also pegylated to improve its biokinetic properties. Anesthetized animals bearing xenografts have been imaged to study tumor uptake at different time points. Biodistribution has been studied in animals with tumors and inflammatory lesions.

Consequently, the invention is directed to the use of a peptide comprising the structure CXCXLLGCC, wherein X is any amino acid residue, or its derivative in tumor and inflammation targeting.

Further objects of the invention are the corresponding methods, i.e. a method for imaging tumor cells or infections/inflammation of a patient, and a method for targeting cytotoxic or cytostatic agents to tumor cells.

Another object of the invention is a diagnostic composition comprising at least one peptide comprising the structure CXCXLLGCC, wherein X is any amino acid residue, or its derivative.

In a preferred embodiment the peptide used in the invention is a peptide comprising the structure CPCPLLGCC or its derivative.

In a method for the treatment of acute myeloid leukemia (AML), an effective amount of a pharmaceutical composition comprising a) a therapeutical agent, preferably an anthracycline; b) a peptide comprising the structure CXCXLLGCC, wherein X is any amino acid residue, or its derivative; and optionally c) conventional pharmaceutically acceptable carriers, excipients and auxiliary agents; is administered to a patient in need of such a treatment.

### BRIEF DESCRIPTION OF THE FIGURES

FIG. 1 demonstrates tumor targeting in human myelomonocytic leukemia in a mouse model. Using metal chelation, example In-111.

FIG. 2 demonstrates tumor targeting at 24 hrs after intravenous I-125-YADGA LLG peptide injection.

FIG. 3 shows tumor targeting at 24 hrs after intravenous PEGylated I-125-YADGA LLG peptide injection.

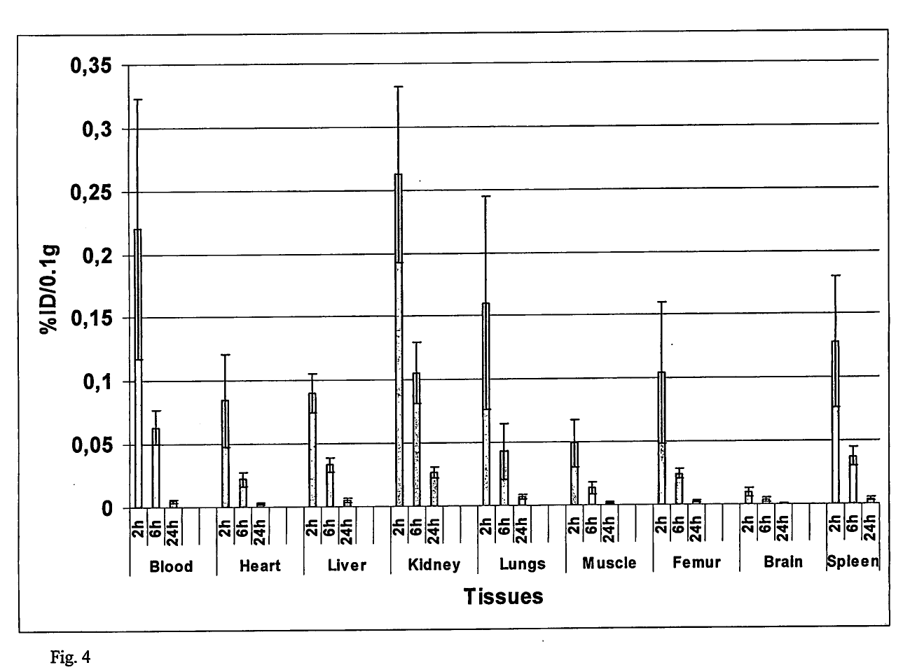

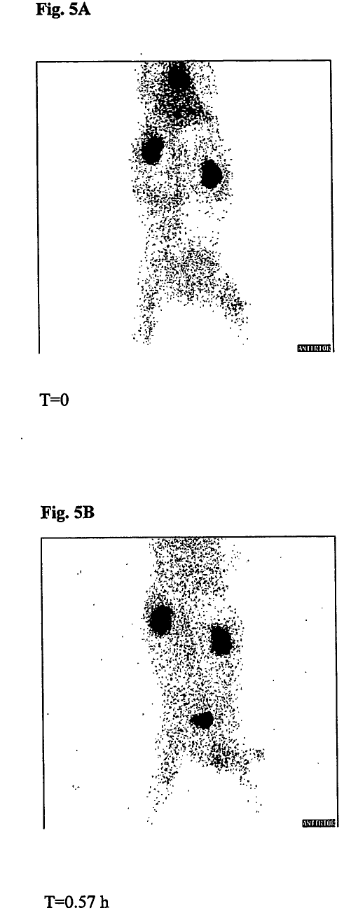

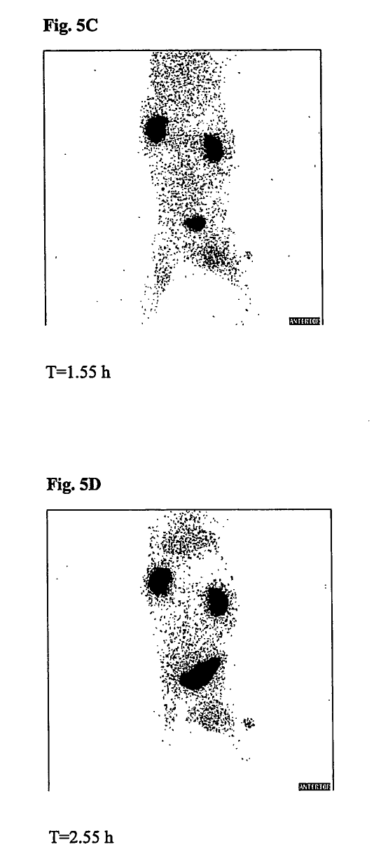

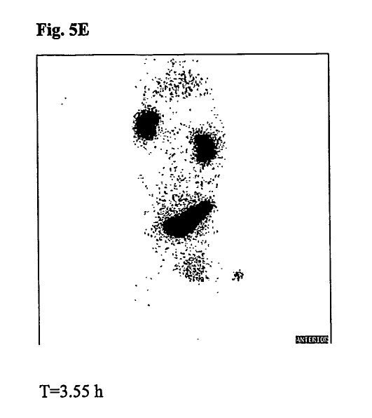

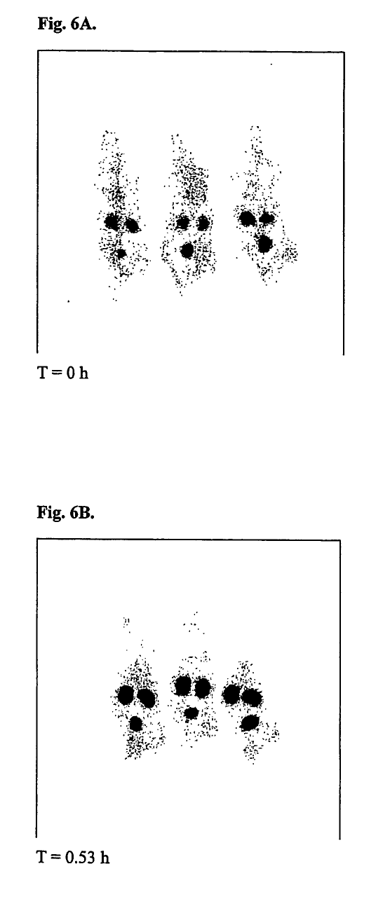

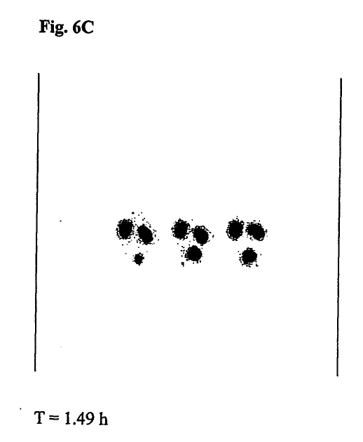

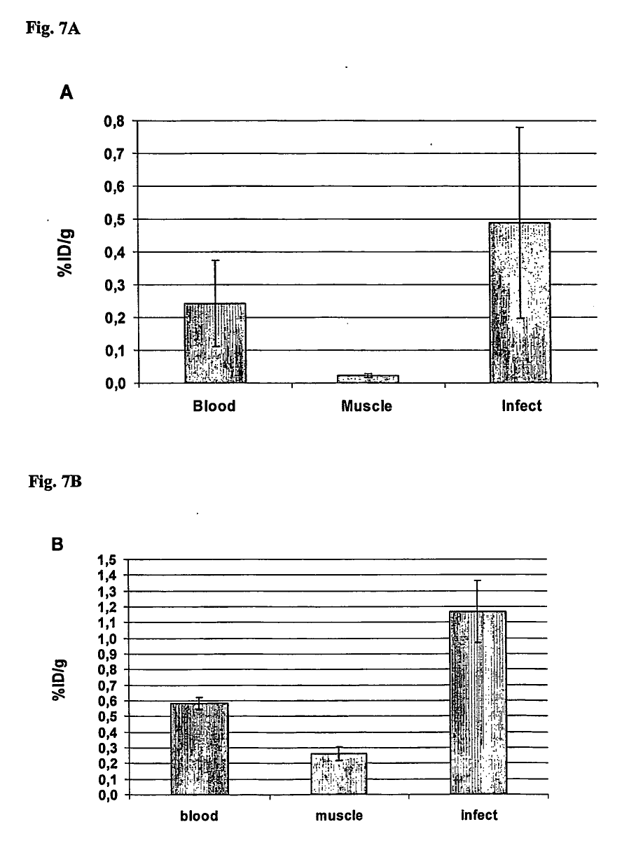

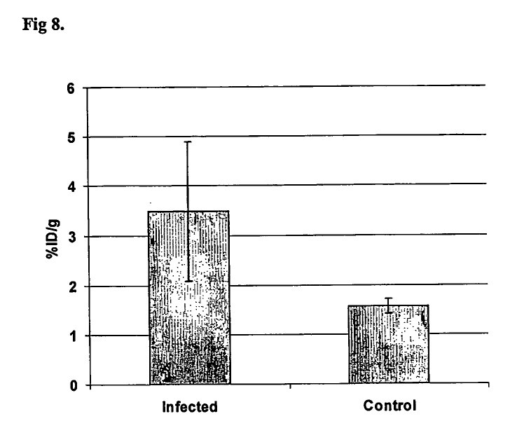

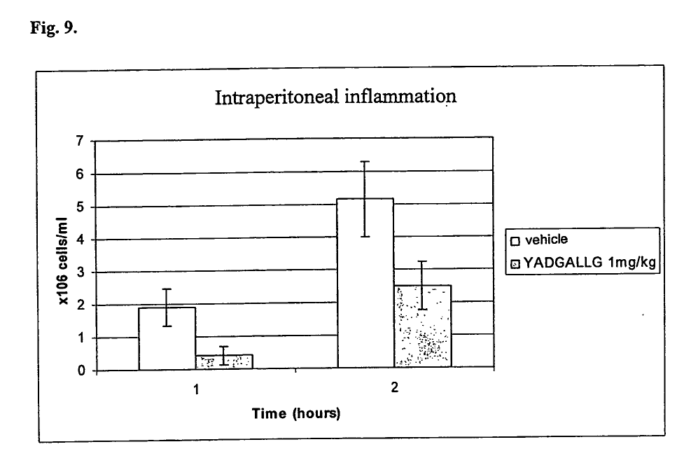

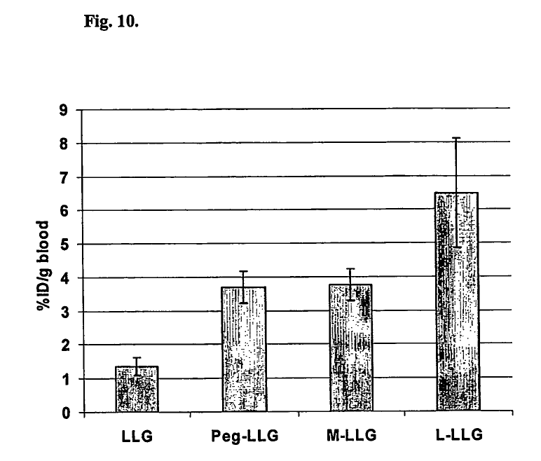

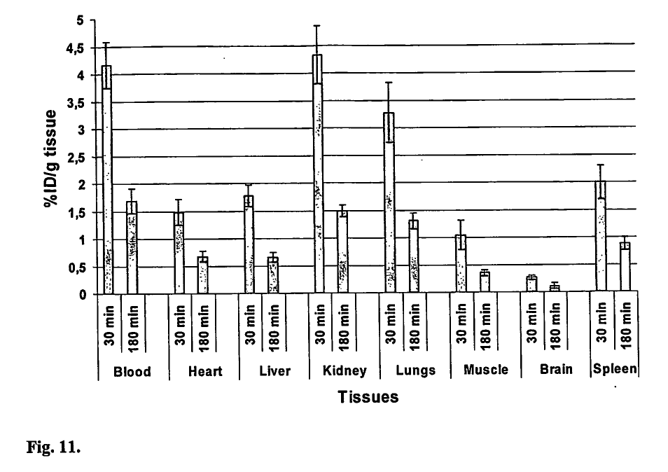

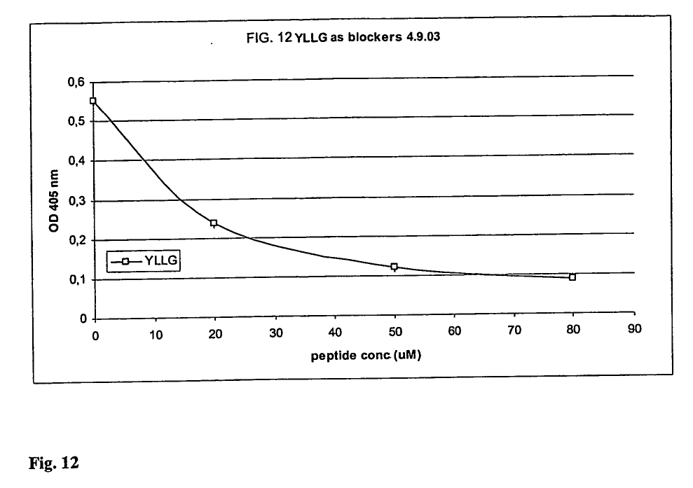

FIGS. 2-3.

Tumor targeting is shown by halogenated LLG-derivatives, left naked peptide, right pegylated peptide. I-125 label, mouse model of human myelomonocytic leukemia. Planar gamma image. In these figures animals have been injected with radiolabelled peptide and anesthesized animals have been imaged under gamma camera (pinhole collimator, Picker SX-300 gamma camera).

FIG. 4 shows the biodistribution study of I-125-YADGA LLG-peptide. The in vivo biodistribution of the ¹²⁵I-labeled-peptide was assessed in NMRI/nude mice at three time points after injections. The biodistribution of the ¹²⁵I-labeled peptide in mice 2 h, 6 h and 24 h p.i. corrected for weight. Results are expressed as percentage of injected dose per 0.1 g tissue (% ID/0.1 g). All values are indicated as mean±SD of 5 mice.

FIGS. 5A-5E show accumulation of the In-111 radiolabeled peptide CPCFLLGCC to an E. coli abscess in the left tight muscle of New Zealand White rabbits.

FIGS. 6A-6C show accumulation of the In-111 radiolabeled peptide CPCFLLGCC to an S. aureus abscess in the left tight muscle of Wistar rats.

FIG. 7A shows biodistribution of In-111-cDTPA-CPCFLLGCC for certain tissues of rabbits, corrected for weight.

FIG. 7B shows biodistribution of In-111-cTPA-CPCFLLGCC for certain tissues of rats, corrected for weight.

FIG. 8 shows accumulation of I-125-GST-LLG in infected mouse ear.

FIG. 9 shows inhibition of leukocyte migration in inflammation by using LLG-peptide.

FIG. 10 shows stability of I-125-LLG conjugates in blood at 3 h p.i.

FIG. 11 shows biodistribution of LLG peptide in mice.

FIG. 12 shows YLLGs capability of blocking LLG-GFPs binding to THP-1 cell line.

### DETAILED DESCRIPTION OF THE INVENTION

Abbreviations:

AML acute myeloid leukaemia

cDTPA cyclic diethylene triamine pentaacetic acid

EDC-NHS ethyldimethylaminopropylcarbodiimide-N-hydroxysuccinimidyl

HPLC high pressure liquid chromatography

HYNIC 6-hydrazopyridine-3-carboxylic acid

LLG CPCFLLGCC

LLG-PEG pegylated CPCFLLGCC

MRI magnetic resonance imaging

NMR nuclear magnetic resonance

PEG polyethylene glycol

PEG-NHS polyethylene glycol-N-hydroxysuccinimidyl

At first, YADGA derivative of LLG peptide was studied for tumor targeting in U937 cell line. The peptide was labelled using In-111 label and direct iodination and cDTPA. Because tumor targeting was successful, further derivatives were developed for further imaging characterization. They were expanded to include Tc-99m and further chelating agents, such as HYNIC. This peptide was also coupled to PEG-NHS with successful imaging.

The inventors found with an animal leukemic model that the LLG can be used as a targeting agent as such, and that it can be modified with PEG-molecule. This is highly important, because these agents are thought to be nontoxic to humans and are easily tested and produced. The invention is also directed to the use of LLG as a targeting agent of cytotoxic or cytostatic agents in liposomes. Further, LLG can improve to control the effect of cytotoxins with less side-effects. This was evaluated in AML with the leukemic animal model. In this form of leukemia the treatment outcome is at the moment unacceptable and new treatment modalities are needed.

LLG is a peptide binding to leukocyte integrins (J Biol Chem 2001; 153:905-15). A YADGACPCFLLGCC derivative was developed for further imaging characterization. Radiolabeling methods for In-111 and I-125 derivatives were developed. The LLG peptide was also coupled to PEG-NHS. Further radionuclide modifications were developed to include also phospholipid linked PEG and liposomal constructs.

The radiolabelled peptide derivatives were imaged at different time points using gamma camera in order to study tumor uptake in vivo as a function of time. After last imaging, tumor tissue were extirpated and counted for radioactivity. Detailed microdistribution was studied using quantitative autoradiography.

YADGA LLG peptide was studied for tumor targeting in human myelomonocytic leukemia U937 cell line. The peptide was labelled using In-111 label and direct iodination, as well as cDTPA.

Peptidoliposomes used for therapeutic approaches could additionally be imaged. Liposomes can be encapsulated with gaseous particles for sonography, paramagnetic compounds for MRI and fluorescein label for fluorescence imaging and e.g. luciferase enzyme system for chemiluminescence imaging.

Later, a liposomal construct which contains anthracycline called idarubicin which is currently most effective treatment of AML, but has toxic effects, as a therapeutic agent and LLG as targeting agent will be developed. The LLG can also function as a therapeutic agent on surface of liposome. Using this labelled liposome we can study the pharmacokinetics and dynamics of idarubicin.

Testing of the LLG constructs in animals provides a background for the clinical development of the treatment of acute myeloid leukemia (AML). An understanding of the cellular pharmacology, cytokinetics and pharmacokinetics of LLG constructs in leukemic mice will show substantial schedule and dose dependency.

Pegylated LLG-Peptide and Liposomes Bearing LLG-Peptide

Pegylation of peptides usually makes them more stabile in serum and therefore more effective. This simple and fast modification of a peptide can make the peptide so stabile in a serum that it can be used as a therapeutic agent and as an imaging agent. To the N-terminus of the LLG-peptide YADGA sequence is added for the labeling procedure, and to have a linkage between the peptide and PEG-molecule. This peptide is coupled to PEG-NHS with different molecular weights with EDC-NHS reaction. To find out the best molecule this construct is tested on cell culture and biodistribution is evaluated on mice bearing xenografts.

Thioglycolate Incubation

Initially, incubation of thioglycolate has been tested in animals after injecting the substance intraperitoneally. Animals are studied for biodistribution at 10 min. Intraperitoneal fluid is collected and cells are stained for integrin expression. Highest uptake is used for further studies. Cytological samples are collected for characterization of neutrophil recruitment at inflammatory sites.

LLG-Peptide Targeting after Thioglycolate Incubation

At one of the selected incubation time points of thioglycolate to develop relevant inflammation, detailed biodistribution study of labelled LLG-peptide construct was performed. Peptide uptake was studied at different time points: 5 min, 30 min, 3 hr, 18 hr after injection. Special attention was paid to intraperitoneal fluid collections. Cytological samples were collected for evaluating neutrophil recruitment at inflammatory sites.

LPS Incubation

Initially, incubation of LPS is tested in animals. Incubation times of 72 hours, 24 hours and 3 hours were tested. Animals were studied for biodistribution at 3 hours. Highest uptake at 24 hrs was used for further studies.

LLG-Peptide Targeting after LPS Incubation

At one of the selected incubation time points of LPS to develop relevant inflammation, detailed biodistribution study of labelled LLG-peptide construct was done. Histological samples were collected for evaluating neutrophil recruitment at inflammatory sites. Normal biodistribution data using iodinated peptide is shown in FIG. 4

Results

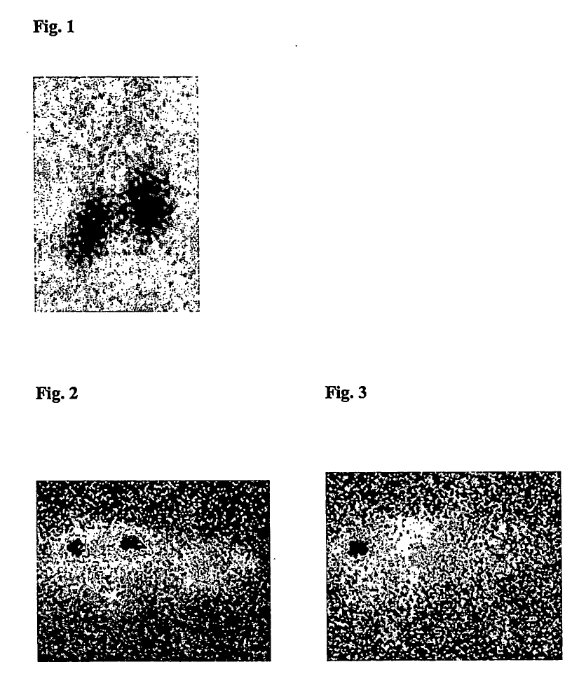

YADGA LLG peptide was studied for tumor targeting in U937 cell line. The peptide was labelled using In-111 label and direct iodination and cDTPA. FIG. 1 demonstrates clearly tumor targeting at 3 hrs after intravenous In-111-YADGA LLG injection. In this model absolute tumor-to-blood ratio was 4.7 at 24 hrs.

We have demonstrated radiohalogenation of LLG and pegylated LLG. Halogenation can be performed similarly using radionuclides I-123, this isotope can also be used for gamma images, and I-124 which could be utilized for positron emission tomography, (images), and I-125 (Auger-therapy, gamma probe, operation techniques), and I-131 (gamma images, radionuclide therapy, beta radiation). Furthermore possible useful radionuclides are Br-76, Br-77, At-211. Bromine is a positron emitter and astatine an alpha-emitter (radionuclide therapy).

In-111 is a transition metal. The same method could be used for radiolabelling of numerous radiometals.

Metallic radionuclides with cDTPA chelation described are In-111, other examples In-110 (PET), In-114m (Auger, gamma) etc. Other similar are Y-90 and other nuclides, Co, Fe, Ni, Cu, Zn, basically all transition metals and their radionuclides. Gd is the metal used for paramagnetic contrast agents, and it can be coupled with cDTPA chelation. Most of lanthanides have characteristics useful for paramagnetic imaging and cDTPA chelation can be utilized.

We have also used peptidoliposomes for imaging. Liposomes can be encapsulated with gaseous particles for ultrasonography, paramagnetic compounds for MRI and fluorescein label for fluorescence imaging and e.g. luciferase enzyme system for chemiluminescence imaging.

In the following experiments, in radiolabeling either the longer construct of the peptide (YADGACPCFLLGCC), shorter version (CPCFLLGCC) or fusion protein GST-LLG was used, depending on the labelling method.

LLG Targeting to Abscess

In this experiment, the targeting of LLG to sites of inflammation was examined in Wistar rats and New Zealand White rabbits, by inducing an abscess with approximately 1×10⁹ colony-forming units of Staphylococcus aureus or Escherichia coli injected into the left tight muscle. During the procedure, the animals were anesthetized. After 24 hours, when swelling of the muscle was apparent, the In-111 radiolabeled peptide CPCFLLGCC was injected i.v. and accumulation of the peptide was followed with gamma camera imaging. The peptide was tested using 3 animals in both animal species.

Although the method was not optimized, the LLG imaging (gamma camera) of the E. coli abscess in rabbits demonstrated specific targeting into tight muscle, which was clearly visible within one hour (see FIG. 5 A-E). The animals were followed-up for 4 hours. In late images urinary excretion disturbed the imaging, but signal-to-background ratio remained high. No other targets than abscess could be detected. Imaging would have been even more successful, if the rabbits would have been catheterized (or bladder emptied) before imaging session. This peptide is highly hydrophilic but easy to label, and it showed rapid clearance through kidneys.

In rats assay, animals were followed-up for 2 hours after peptide injection. LLG imaging using gamma camera of the S. aureus abscess demonstrated also specific targeting (FIG. 6 A-C), but in late images urinary excretion disturbed the imaging. Similarly, in rats the demonstration of targeting would have been more effective if the bladders had been emptied before imaging. However, signal-to-background ratio remained high, and as in rabbits, no other targets than abscess could be detected.

After the imaging period, animals were sacrificed, various tissues were collected and the accumulated radioactivity was measured using a gamma-counter. In FIG. 7A, the amount of accumulated peptide (expressed as percentage of injected dose/weight of the tissue measured; % ID/g) is shown for certain tissues of rabbits (mean of 3 animals). No organ (except kidney, data not shown) showed as high accumulation as the abscess, in which the accumulation was 21.7-fold when compared to muscle, and 2.3-fold when compared to blood. FIG. 7B shows the same accumulation measured from rats. In these animals, the corresponding ratios were 4.5 and 2.0 for muscle and blood, respectively. These experiments clearly show that radioactively labelled LLG is an efficient means of imaging infection sites, but due to its fast clearance, no urinary tract infections can be detected with this construct.

LLG Targeting to Sites of Inflammation (Ear)

In this experiment, six Balb/c mice were injected in their left ear with 10 μg of E. coli LPS. Inflammation was developing for 24 h, then 20 μg (75 kBq) of radiolabeled GST-LLG was injected into tail vein of the mice. At 3 h after peptide injection the mice were sacrificed and the left ears (infected) and right ears (control) were collected to measure the accumulated radioactivity. Results are expressed as percentage of injected dose per 1.0 g tissue (% ID/g) (FIG. 8). All values are indicated as the mean±SD of 3 mice.

LLG Induced Inhibition

In our previous studies (supra), we have shown that the administration of LLG-GST fusion protein intravenously into mice inhibited the migration of neutrophils into thioglycolate inflamed peritoneal cavity. The number of migrating neutrophils was reduced to 40% of control. This effect was time dependent and visible in time points 1 h and 2 h, decaying with time and not visible at 4 hours and later.

The study was repeated as follows:

To induce inflammation, female Balb/c mice were injected intraperitoneally with 1 ml of 3% Thioglycolate Broth (TG), three animals/group. Mice in the control group were injected iv with plain vehicle PBS-10% DMSO, and mice in the peptide group were injected iv with 1 mg/kg YADGACPCFLLGCC in PBS-10% DMSO. After 60 or 120 minutes, the mice were sacrificed. Cells in peritoneal cavity were collected by lavage with 5 ml PBS-5 mM EDTA, and counted with a hemocytometer.

The local injection of TG has been shown to cause a significant extravasation of polymorphonuclear leukocytes into the cavity. In this experiment different cell populations were not distinguished. However, once again, YADGACPCFLLGCC reduced the accumulation of cells in experimental inflammation in vivo by 78% after 60 minutes and 52% after 120 minutes (FIG. 9).

The Stability of LLG

In order to find out the most stable form of the LLG peptide (CPCFLLGCC), the peptide was labelled with I-125. The purified peptide was coupled to PEG₍₁₀₀₀₀₎ or to DSPE-PEG₍₃₄₀₀₎. In water solutions DSPE-PEG₍₃₄₀₀₎-LLG forms micelles, that were incorporated into commercially available stealth liposomes. I-125-LLG (LLG), pegylated LLG (Peg-LLG), micellar LLG (M-LLG) and liposomal LLG (L-LLG) were injected into the tail vein of Balb/c mice. At 3 h after peptide injection, the mice were sacrificed, blood samples were collected and measured for radioactivity. Results are expressed as percentage of injected dose per 1.0 g blood (% ID/g). All values are indicated as mean±SD of 5 mice.

As shown in FIG. 10, coupling of the peptide to a higher molecular weight molecule or to a stealth liposome, increases the stability of the peptide in circulation up to 7-fold.

Biodistribution of LLG

LLG (YADGACPCFLLGCC) was labelled with I-125, and the purified peptide (40 μg; ˜500 kBq) was injected into the tail vein of mice in the volume of 100 μg. At 30 min and 180 min after peptide injection, the mice were sacrificed and their blood and tissues were collected to measure the radioactivity. Results are expressed as percentage of injected dose per 1.0 g tissue (% D/g) (FIG. 11). All values are indicated as the mean±SD of 3 mice.

As shown in FIG. 11, the peptide did not accumulate in any tissue, and a rapid clearance through kidneys could be seen.

The Affinity of LLG

We examined the affinity of LLG to integrin using BIACORE. In this method, purified integrin I-domain was immobilized on the gold coated carboxymethylated dextran chip. The immobilization succeeded, and in various channels a RU between 2000-4000 was obtained. Various concentrations (3.3 nM-33 μM) of LLG-GST or GST were tested for their ability to bind to the I-domain. Reaction buffer was 10 mM HEPES (pH 7.4)-150 mM NaCl, with or without 1 mM MgCl₂. Unfortunately, no specific binding could be detected for LLG, because GST protein caused a very high background binding. Addition of detergent P20 0.05% could not diminish the background.

In another set of experiment, plain LLG peptide was used for affinity testing. The concentration of the tested peptide varied between 134 nM-134 μM. Under the above described set up, no specific binding could be detected, due to the small size of the peptide. The BIACORE method is currently under development, and we intend to study the affinity again with a peptide coupled to a higher molecular weight, inert carrier molecule.

FIG. 12 shows YLLGs capability of blocking LLG-GFPs binding to THP-1 cell line. What has been observed is that at 50 μM YLLG concentration 95% of LLG-GFPs binding is been blocked. When concentrations are been lowered to 20 μM still 70% inhibition occurs. Based on the FIG. 12 it is evident that the IC 50 is on nanomolar scale. However, due to the unspecific binding of peptide to the plastic walls of the container and the relative high concentrations of LLG-GFP needed for signal nanomolar scale, experiments can not been performed with this setup on its current already un-optimized state. Although these experiments do not give binding constant directly they actually tell from peptides capability to bind in biological systems which is more relevant in in vivo systems.

1. Use of a peptide comprising the structure
CXCXLLGCC
wherein X is any amino acid residue, in tumor and/or infection targeting.

2. Use of a peptide comprising the structure
CXCXLLGCC
wherein X is any amino acid residue, for diagnostic purposes wherein the peptide is coupled to a radioactive label, an affinity label, a magnetic particle, a fluorescent or luminescent label, for use as an imaging agent.

3. Use of a peptide comprising the structure
CPCFLLGCC
or in tumor and infection targeting.

4. Use of a peptide comprising the structure
CPCFLLGCC
for diagnostic purposes wherein the peptide is coupled to a radioactive label, an affinity label, a magnetic particle, a fluorescent or luminescent label, for use as an imaging agent.

5. Use according to claim 1 or 3, wherein the peptide is used as a targeting agent of cytotoxic or cytostatic agent.

6. Use according to claim 1, wherein the peptide has been modified with PEG-molecule.

7. Use according to claim 1, wherein the peptide is coupled to a liposome.

8. A method for imaging tumor cells or infections/inflammation of a patient comprising the steps of
(a) coupling a detectable label with at least one peptide compound selected from the group consisting of peptides having the structure
CXCXLLGCC
wherein X is any amino acid residue,
(b) administering the mixture obtained to a patient, and
(c) detecting the label.

9. The method according to claim 8, wherein the detectable label is selected from the group consisting of a radioactive label, an affinity label, a magnetic particle, a fluorescent label and a luminescent label.

10. A method of targeting and imaging tumor cells or infections/inflammation comprising administering a composition comprising at least one compound selected from the group consisting of peptides having the structure
CXCXLLGCC
wherein X is any amino acid residue, a detectable label, and optionally a chemotherapeutic or anti-inflammatory agent, to a patient suspected of having a tumor or infection; and
if appropriate, detecting the label.

11. A kit for imaging tumor cells in a patient, comprising
at least one peptide compound selected from the group consisting of peptides having the structure
CXCXLLGCC
wherein X is any amino acid residue, and
a detectable label.

12. The kit according to claim 11, wherein the detectable label is selected from the group consisting of a radioactive label, an affinity label, a magnetic particle, a fluorescent label and a luminescent label.

13. A diagnostic composition comprising at least one peptide comprising the structure
CXCXLLGCC
wherein X is any amino acid residue, or its derivative, optionally together with diagnostically acceptable carriers, excipients, and auxiliary agents.

14. The diagnostic composition according to claim 13, wherein the peptide comprises the structure
CPCFLLGCC.

15. (canceled)
